(19) United States
(12) Patent Application Publication
(10) Pub. No.: US 2016/0377596 A1
(43) Pub. Date: Dec. 29, 2016

(54) EXHALATION DIAGNISTIC APPARATUS

(71) Applicant: Kabushiki Kaisha Toshiba, Minato-ku (JP)

(72) Inventors: Miyuki KUSABA, Meguro (JP); Akira MAEKAWA, Kamakura (JP); Shigeyuki TAKAGI, Fujisawa (JP); Tsutomu KAKUNO, Fujisawa (JP); Hiroshi HASEGAWA, Yokosuka (JP); Yasutomo SHIOMI, Koza (JP)

(73) Assignee: Kabushiki Kaisha Toshiba, Minato-ku (JP)

(21) Appl. No.: 15/260,436

(22) Filed: Sep. 9, 2016

Related U.S. Application Data
(63) Continuation of application No. PCT/JP2015/057701, filed on Mar. 16, 2015.

(30) Foreign Application Priority Data
Sep. 22, 2014 (JP) 2014-192320

(51) Int. Cl. G01N 33/497; G01N 21/39; G01N 21/3504; A61B 5/08

(52) U.S. Cl. CPC G01N 33/497; A61B 5/082; G01N 21/39; G01N 21/3504; A61B 2010/0087

(57) ABSTRACT

An exhalation diagnostic devise includes a cell portion, a light source, a detector and a controller. The cell portion includes space into which a sample gas containing first and second substances is introduced. The detector detects an intensity of the light transmitted through the space. The controller, at a time of a first operation, causes the light source to change a wavelength, and calculates a ratio of an amount of the second substance to an amount of the first substance. And the controller, at a time of a second operation performed in one respiration, causes the light source to set the wavelength of the light to a third wavelength, determines whether concentration of at least one of the first substance and the second substance exceeds a set value or not, and starts the first operation when the concentration exceeds the set value.

### CROSS-REFERENCE TO RELATED APPLICATIONS

This is a continuation application of International Application PCT/JP2015/057701, filed on Mar. 16, 2015. This application also claims priority to Japanese Application No.2014-192320, filed on Sep. 22, 2014. The entire contents of each are incorporated herein by reference.

### FIELD

Embodiments described herein relate generally to a exhalation diagnostic apparatus.

### BACKGROUND

Gas in exhaled air is measured in exhalation diagnostic devices. Prevention and early detection of disease is facilitated by the results of such measurements. It is desired that highly accurate measurement results are obtained in exhalation diagnostic devices.

### BRIEF DESCRIPTION OF THE DRAWINGS

FIG. 1 is a schematic view illustrating an exhalation diagnostic device according to a first embodiment;

FIGS. 2A and 2B are schematic drawings illustrating examples of the exhalation diagnostic device according to the first embodiment;

FIGS. 3A and 3B are schematic drawings illustrating examples of the exhalation diagnostic device according to the first embodiment;

FIGS. 4A and 4B are graphs illustrating characteristics of carbon dioxide;

FIG. 5 is a schematic view illustrating the exhalation diagnostic device according to the first embodiment;

FIG. 6 is a schematic view illustrating the operations of the exhalation diagnostic device according to the first embodiment;

FIG. 7 is a schematic view illustrating an exhalation diagnostic device according to a second embodiment; and

FIGS. 8A to 8C are schematic views illustrating a portion of the exhalation diagnostic device according to an embodiment.

### DETAILED DESCRIPTION

In general, according to one embodiment, an exhalation diagnostic devise includes a cell portion, a light source, a detector and a controller. The cell portion includes space into which a sample gas containing a first substance and a second substance different than the first substance is introduced. The light source emits light toward the space. The detector detects an intensity of the light transmitted through the space. The controller, at a time of a first operation, causes the light source to change a wavelength of the light within a wavelength band including a first wavelength of a first peak of light absorption of the first substance, and a second wavelength, differing from the first wavelength, of a second peak of light absorption of the second substance, and calculates a ratio of an amount of the second substance contained in the sample gas to an amount of the first substance contained in the sample gas on a basis of detection results of an intensity of the light of the first wavelength and an intensity of the light of the second wavelength detected by the detector. And the controller, at a time of a second operation performed in one respiration, causes the light source to set the wavelength of the light to a third wavelength, determines whether concentration of at least one of the first substance and the second substance exceeds a set value or not on a basis of detection results of the intensity of the light of the third wavelength detected by the detector, and starts the first operation when the concentration exceeds the set value.

### First Embodiment

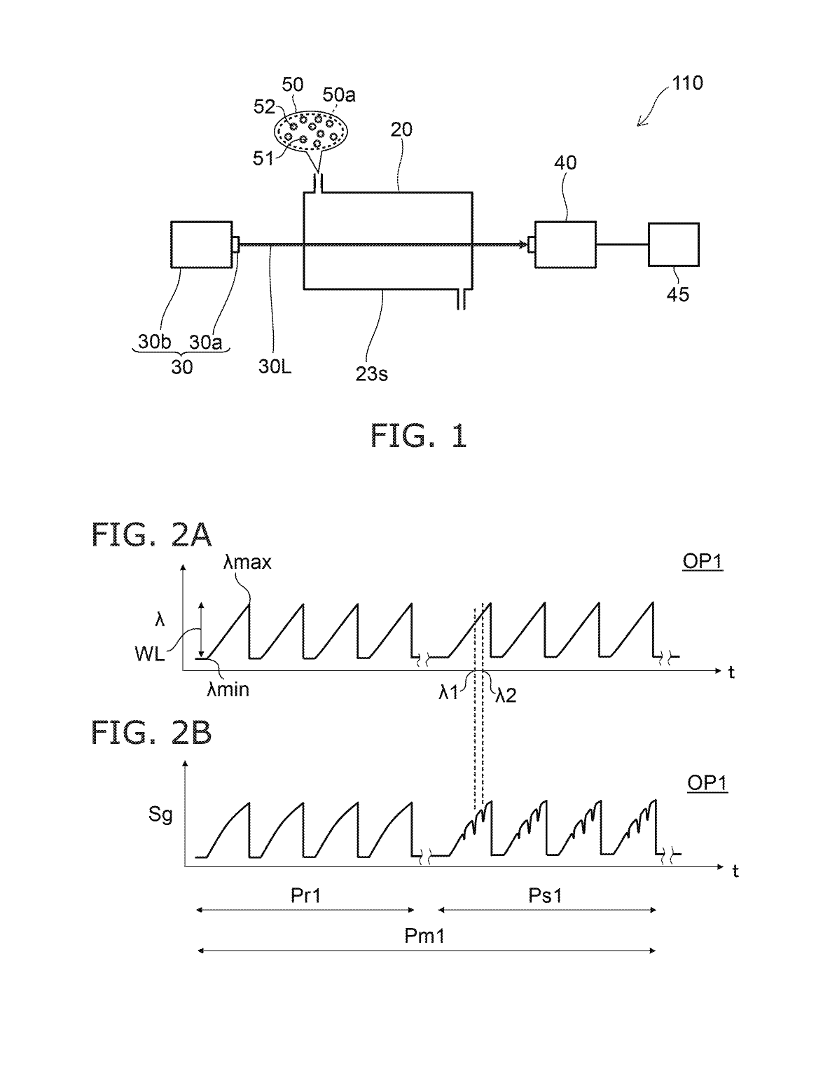

FIG. 1 is a schematic view illustrating an exhalation diagnostic device according to a first embodiment.

As illustrated in FIG. 1, an exhalation diagnostic device 110 according to the embodiment includes a cell portion 20, a light source 30, a detector 40, and a controller 45.

A sample gas 50 is introduced into the cell portion 20. Specifically, the sample gas 50 is introduced into a space 23s provided in the cell portion 20. The sample gas 50 contains a first substance 51 and a second substance 52. The second substance 52 differs from the first substance 51.

The sample gas 50 contains exhaled air 50a. The exhaled air 50a is exhaled air of, for example, an animal, including humans. The exhaled air 50a contains carbon dioxide including ¹²C (¹²CO₂) and carbon dioxide including ¹³C (¹³CO₂). These carbon dioxides may include isotopes of oxygen.

The first substance 51 is the carbon dioxide including ¹²C (¹²CO₂). The second substance 52 is the carbon dioxide including ¹³C (¹³CO₂). The embodiment is not limited thereto, and the first substance 51 and the second substance 52 may be other substances. In the following, a case is described where the first substance 51 is the carbon dioxide including ¹²C (¹²CO₂) and the second substance 52 is the carbon dioxide including ¹³C (¹³CO₂).

There is a relationship between a health condition of a human and a ratio of the isotopes of carbon (¹²CO₂ and ¹³CO₂) in the carbon dioxide contained in the exhaled air 50a. The health condition of a human can be diagnosed by that human drinking a labeled compound of concentrated ¹³C (a ¹³C labeled compound). For example, the human drinks ¹³c-urea as the ¹³C labeled compound. In this case, the relative amount of the ¹³CO₂ will increase if helicobacter pylori is present. Alternatively, for example, the human drinks ¹³C-acetate as the ¹³C labeled compound. In this case, gastric emptying can be diagnosed by evaluating the exhaled air 50a. There is a relationship between gastric emptying and the relative amount of ¹³CO₂ in cases where the ¹³C-acetate is drunk. As described later, light absorption of the first substance 51 (¹²CO₂) has a first peak in a first wavelength. Light absorption of the second substance 52 (¹³CO₂) has a second peak in a second wavelength. By using light of a wavelength corresponding to the wavelengths of these two peaks, amounts of the first substance 51 and the second substance 52 (relative proportions) can be detected.

The light source 30 incidents light (measurement light 30L) into the space 23s. The light source 30 can change the wavelength of that light (the measurement light 30L). As described later, the changing of the wavelength is performed in a specific wavelength band. This wavelength band includes the first wavelength of the first peak of the light absorption of the first substance 51, and the second wavelength of the second peak of the light absorption of the second substance 52.

In this example, the light source 30 includes a light emitting portion 30a and a driving portion 30b. The driving portion 30b is electrically connected to the light emitting portion 30a. The driving portion 30b supplies power to the light emitting portion 30a for light emission. As described later, a distributed feedback (DFB type) quantum cascade laser, for example, is used as the light emitting portion 30a. An interband cascade laser (ICL) may also be used as the light emitting portion 30a. An example of the light emitting portion 30a is described later.

The measurement light 30L passes through the space 23s of the cell portion 20. A portion of the measurement light 30L is absorbed by the substances contained in the sample gas 50 (the first substance 51 and the second substance 52). Of the measurement light 30L, components of wavelengths specific to these substances are absorbed. The degree of absorption is dependent on the concentration of the substances.

The detector 40 detects, for example, the measurement light 30L that has passed through the space 23s in a state where the sample gas 50 is introduced to the space 23s. The detector 40 detects an intensity of the light (the measurement light 30L) that has passed through the space 23s. A detection device 41 having sensitivity in the infrared region is used in the detector 40. For example, a thermopile, a semiconductor sensor device (e.g. InAsSb), or the like is used for the detection device 41. The detector 40 may be provided with a circuit portion 42 that processes signals output from the detection device 41. In this embodiment, the detector 40 is optional.

In the detector 40, in addition to the intensity of the light when the sample gas 50 is introduced to the space 23s, the intensity of the light intensity when the sample gas 50 has not been introduced to the space 23s is also detected. The latter is used as a reference value in the detection. Moreover, for example, the detection described above is performed multiple times. Specifically, the detector 40 performs, multiple times, an operation including a first detection of the intensity of the light (the measurement light 30L) that has passed through the space 23s in which the sample gas 50 has been introduced and a second detection of the intensity of the light (the measurement light 30L) that has passed through the space 23s in which the sample gas 50 has not been introduced.

The controller 45 calculates a ratio of the amount of the second substance 52 to the amount of the first substance 51 in the sample gas 50 on the basis of results obtained through the multiple performances of the operation described above. Specifically, the controller 45 calculates the ratio of the amount of the second substance 52 to the amount of the first substance 51 on the basis of results of a plurality of first detections obtained by the multiple performances of the operation described above and results of a plurality of second detections obtained by the multiple performances of the operation described above. Thus, in the exhalation diagnostic device 110, the amount of the second substance 52 contained in the exhaled air 50a (the sample gas 50) can be identified, and a highly accurate diagnosis can be performed.

Here, in respiration, inhalation and exhalation are repeated. This repetition is repeated at a frequency of about 20 times per minute. Depending on the timing of the measurement of the exhaled air, the sample gas 50 may contain not only the exhaled air 50a, but also a large amount of air. In such a case, accurate measuring is difficult. Thus, it is preferable that the measurement be performed in a state where the proportion of the amount of the exhaled air 50a in the sample gas 50 is high.

For example, changes over time of an amount (proportion) of a target substance (e.g. carbon dioxide) contained in the sample gas 50 is monitored and, where that amount (proportion) exceeds a reference value, the first detection and the second detection described above are begun. Thus, the measurement will be performed in a state where the proportion of the amount of the exhaled air 50a in the sample gas 50 is high, and a highly accurate diagnosis is possible.

Such monitoring can be performed by the exhalation diagnostic device 110. Specifically, the controller 45 is capable of performing a first operation and a second operation. In the first operation, the first detection and the second detection described above are performed, and the ratio of the amount of the second substance 52 to the amount of the first substance 51 is calculated. On the other hand, in the second operation, the temporal change of the amount of the target substance (e.g. carbon dioxide) is monitored. Then, the first operation is started on the basis of the results of the monitoring.

For example, a first valve V1 is provided on an inflow port of the cell portion 20 and a second valve V2 is provided on an outflow port of the cell portion 20. In the second operation, these valves are set to an open state. Thus, in the second operation, the sample gas 50 flows into the cell portion 20 and the temporal change of the amount (the proportion) of the target substance (e.g. carbon dioxide) contained in the sample gas 50 is monitored. On the other hand, in the first operation, these valves are set to a closed state. Thus, the flow of the sample gas 50 in the cell portion 20 ceases, and the state of the air flow in the cell portion 20 stabilizes. As a result, highly accurate measuring can be stably performed in the first operation.

FIGS. 2A and 2B are schematic drawings illustrating examples of the exhalation diagnostic device according to the first embodiment.

These drawings illustrate examples of a first operation OP1.

FIG. 2A illustrates an example of changes of the wavelength of the measurement light 30L emitted from the light source 30. FIG. 2B illustrates an example of changes of a signal detected in the detector 40. In the graphs, time t is shown on the horizontal axes. In FIG. 2A, a wavelength λ is shown on the vertical axis. In FIG. 2B, a strength Sg of the signal is shown on the vertical axis.

As illustrated in these graphs, a reference data measurement period Pr1 and a sample data measurement period

Ps1 are provided. In the reference data measurement period Pr1, the sample gas 50 has not been introduced into the space 23s. In the sample data measurement period Ps1, the sample gas 50 is introduced into the space 23s.

In the reference data measurement period Pr1, the wavelength of the measurement light 30L emitted from the light source 30 changes. The changing of the wavelength is performed within a specific wavelength band WL. This wavelength band WL includes a first wavelength λ1 corresponding to the peak of the absorption of the first substance 51 and a second wavelength λ2 corresponding to the peak of the absorption of the second substance 52. The wavelength band WL is, for example, from 4.3573 μm to 4.3535 μm. A difference between a longest wavelength λmax and a shortest wavelength λmin among the wavelength band WL is, for example, for example, approximately 0.0038 micrometers. For example, the difference is 0.003793904 micrometers.

The changing of the wavelength of the measurement light 30L is repeated multiple times. The intensity of the measurement light 30L is detected by the detector 40. The strength Sg of the signal is detected multiple times in the detector 40.

In the sample data measurement period Ps1, the sample gas 50 is introduced into the space 23s, and a portion of the measurement light 30L is absorbed by the first substance 51 and the second substance 52. For example, the strength Sg of the signal is less at the first wavelength λL corresponding to the peak of the absorption of the first substance 51. For example, the strength Sg of the signal is less at the second wavelength λ2 corresponding to the peak of the absorption of the second substance 52.

By comparing the strength Sg (reference strength) of the signal in the reference data measurement period Pr1 with the strength Sg (sample strength) of the signal in the sample data measurement period Ps1, a value corresponding to the amount of the first substance 51 and a value corresponding to the amount of the second substance 52 can be obtained. For example, a ratio of the sample strength to the reference strength is found. For example, a difference between the sample strength and the reference strength is found. As a result, the value corresponding to the amount of the first substance 51 and the value corresponding to the amount of the second substance 52 can be obtained. The ratio of the amount of the second substance 52 to the amount of the first substance 51 can be obtained.

One instance of the measurement (the calculation of the ratio of the amount of the second substance 52 to the amount of the first substance 51) is performed using at least one reference data measurement period Pr1 and at least one sample data measurement period Ps1. That is, in the first operation OP1, one measurement period (a first measurement period Pm1) consists of at least one reference data measurement period Pr1 and at least one sample data measurement period Ps1.

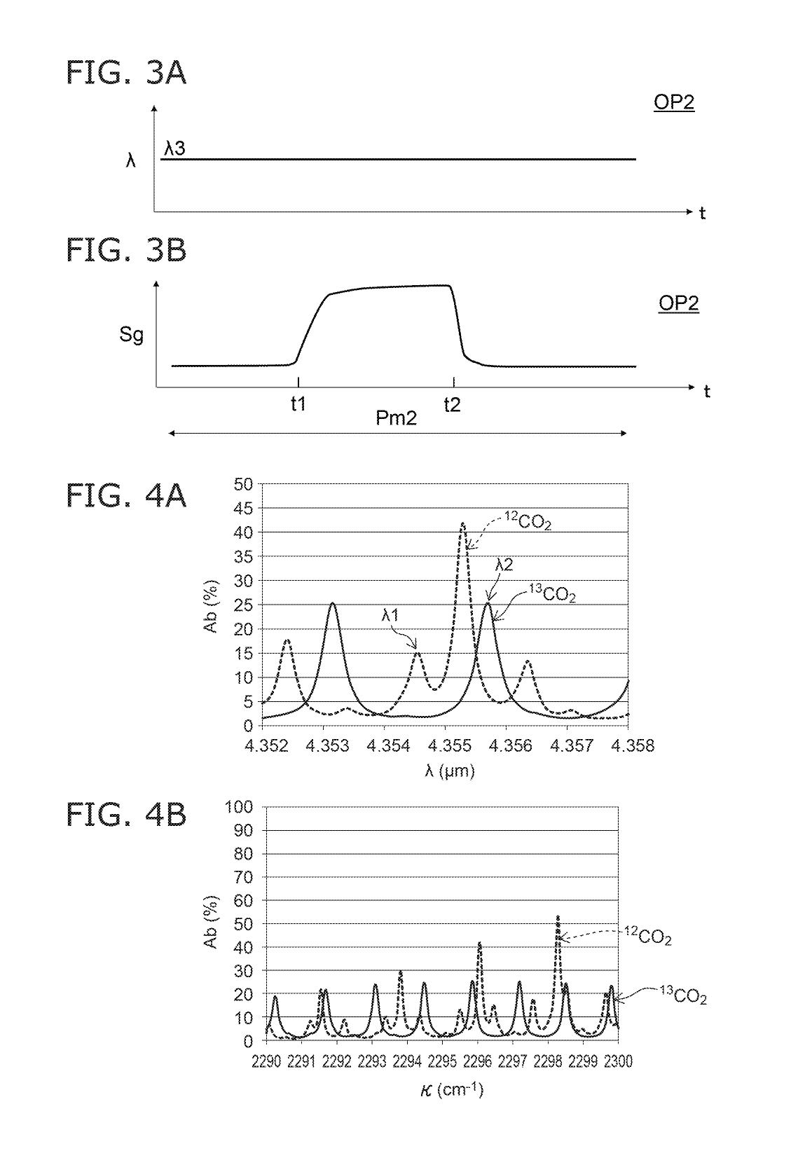

FIGS. 3A and 3B are schematic drawings illustrating examples of the exhalation diagnostic device according to the first embodiment.

These drawings illustrate examples of a second operation OP2.

FIG. 3A illustrates the wavelength of the measurement light 30L emitted from the light source 30. FIG. 3B illustrates an example of changes of a signal detected in the detector 40. In the graphs, time t is shown on the horizontal axes. In FIG. 3A, the wavelength λ is shown on the vertical axis. In FIG. 3B, the strength Sg of the signal is shown on the vertical axis.

As illustrated in FIG. 3A, in this example, in the second operation OP2, the wavelength of the measurement light 30L is substantially constant at a third wavelength λ3. As described later, the third wavelength λ3 may be swept.

As illustrated in FIG. 3B, the strength Sg of the signal begins to change at a time t1, and the strength Sg of the signal reaches a maximum at a time t2. A period before the time ti is a period when the sample gas 50 that has not been substituted by the lungs is introduced into the space 23s of the cell portion 20. In other words, substantially, the space 23s is filled with air. In this state, the substances contained in the air (the amount of carbon dioxide) is detected. At the time ti, air that has been substituted by the lungs begins to be introduced into the space 23s and the strength Sg of the signal begins to increase. At the time t2, the strength Sg of the signal is substantially the strongest. This state corresponds to the state where the exhaled air 50a sufficiently substituted by the lungs is introduced into the space 23s of the cell portion 20.

A time t3 described below may be used in place of the time t2 where the signal (strength Sg) is strongest. For example, the time t3 may be set as a time (a first standard) at which the signal (the strength Sg) becomes a predetermined value. The time t3 may be set as a time (a second standard) at which the changing of the signal (the strength Sg) becomes saturated and a rate of change of the signal (the strength Sg) becomes a predetermined value. The time t3 may be set as a time (a third standard) at which both the first standard and the second standard are fulfilled. The time t3 is defined as a time corresponding to the state where the exhaled air 50a sufficiently substituted by the lungs is introduced into the space 23s of the cell portion 20. In the second operation OP2, a measurement period (a second measurement period Pm2) corresponds to, for example, a time of one respiration.

By starting the first operation OP1 described above when the state of this time t3 is realized, highly accurate measurement of the target substance (the first substance 51 and the second substance 52) can be performed in the state where the exhaled air 50a sufficiently substituted by the lungs is introduced into the space 23s of the cell portion 20.

Such operations can be performed by the controller 45.

That is, the controller 45 performs the following at the time of the first operation OP1.

The controller 45 causes the light source 30 to change the wavelength of the light (the measurement light 30L) within the wavelength band WL that includes the first wavelength λ1 of the first peak of the light absorption of the first substance 51 and the second wavelength λ2, different from the first wavelength λ1, of the second peak of the light absorption of the second substance 52. Then, the controller 45 calculates the ratio of the amount of the second substance 52 contained in the sample gas 50 to the amount of the first substance 51 contained in the sample gas 50 on the basis of the detection results of the intensity of the light of the first wavelength λ1 and the detection results of the intensity of the light of the second wavelength λ2 detected by the detector 40.

Furthermore, the controller 45 performs the following at the time of the second operation OP2.

The controller 45 causes the light source 30 to change the wavelength of the light of the third wavelength λ3. Then, the controller 45 detects the temporal change of the amount of at least either of the first substance 51 and the second substance 52 on the basis of the detection results of the intensity of the light of the third wavelength λ3 detected by the detector 40.

Then, the controller 45 performs the first operation OP1 on the basis of the results of the detection of the temporal change described above. According to the embodiment, a highly accurate exhalation diagnostic device can be provided.

The exhalation diagnostic device 110 can, for example, perform a carbon dioxide monitoring operation (the second operation OP2) and a measurement operation of the first substance and the second substance 52, that is, an isotope ratio measurement operation (the first operation OP1). For example, with the exhalation diagnostic device 110, for example, an operation as a capnometer, and a carbon dioxide isotope ratio measurement operation can be performed.

On the other hand, a reference example is given in which the capnometer operation and the isotope ratio measurement operation are performed separately. In this case, first, in the capnometer operation, carbon dioxide is detected and a valve operation is performed, and a sample gas containing carbon dioxide in abundance is introduced into a cell for isotope ratio measurement. At this time, a time lag occurs due to the time needed to replace the sample gas and the like. Furthermore, there are cases where residual gas in the cell for the isotope ratio measurement is not sufficiently replaced by the target sample gas.

In contrast, in the embodiment, the operation as a capnometer and the carbon dioxide isotope ratio measurement operation are performed using the same cell portion 20. As such, use of the time lag described above is suppressed and the effects of the residual gas are suppressed. Thus, highly accurate isotope measurement is possible.

The time of the exhaled air 50a exhaled in one respiration is not longer than approximately 10 seconds. Accordingly, the second operation OP2 is performed in a time shorter than this time. Specifically, in the second operation OP2, the controller 45 detects the temporal change of at least either the amount of the first substance 51 and the second substance 52 in a period not shorter than 0.3 seconds (the second measurement period Pm2).

Moreover, it is preferable that the measurement in the second operation OP2 is performed substantially continuously within a short period of time. For example, the measurement is performed in a temporal resolution of 0.1 seconds or shorter. Specifically, in the second operation OP2, the controller 45 measures at least either the amount of the first substance 51 and the second substance 52 at a time resolution of 0.1 seconds or shorter, and detects the temporal change of at least either of the amounts. For example, high-speed measurement is possible by combining a quantum cascade laser and a semiconductor sensor device (e.g. InAsSb or the like).

On the other hand, in the first operation OP1, the amounts of the first substance 51 and the second substance 52 are measured with high accuracy. This measurement is performed on the exhaled air 50a supplied in one respiration. For example, the controller 45 continuously performs the first operation OP1 for a period of not shorter than 1 second and not longer than 10 seconds. Thus, highly accurate measurement results can be obtained.

In the embodiment, a capacity of the space 23s provided in the cell portion 20 is preferably not more than 500 cm³ (not more than 500 mL). That is, generally, the capacity (volume) of the exhaled air 50a of one human respiration is not more than 500 mL. As such, by setting the capacity of the cell portion 20 to not more than 500 mL, the cell portion 20 can be filled by the exhaled air 50a of one respiration. Additionally, it is more preferable that the capacity of the cell portion 20 be set to not more than 20 cm³.

By using a quantum cascade laser as the light emitting portion 30a, highly accurate measurement is possible, even when using a cell portion 20 with such a small capacity.

FIGS. 4A and 4B are graphs illustrating characteristics of carbon dioxide.

These graphs illustrate an absorption spectrum of ¹²CO₂ and an absorption spectrum of ¹³CO₂. In FIG. 4A, a wavelength λ (μm) is shown on the horizontal axis. In FIG. 4B, a wavenumber κ (cm⁻¹) is shown on the horizontal axis. Absorptivity Ab (%) is shown on the vertical axes.

As illustrated in FIGS. 4A and 4B, each of ¹²CO₂ and ¹³CO₂ has a unique absorption. For example, there are multiple peaks corresponding to the absorption of ¹²CO₂. Also, there are multiple peaks corresponding to the absorption of ¹³CO₂.

For example, the wavelength of the measurement light 30L emitted from the light source 30 is swept across a range of the wavelength band WL. The wavelength band WL includes a first wavelength λ1 and a second wavelength λ2. The wavelength band WL also preferably includes at least one of either another peak of the absorption of the ¹²CO₂ and another peak of the absorption of the ¹³CO₂.

The first wavelength λ1 is, for example, 4.3553 μm. The second wavelength λ2 is, for example, 4.3557 μm. In the embodiment, the third wavelength λ3 may be set to be substantially the same as the first wavelength λ1. Additionally, the third wavelength λ3 may be set to be substantially the same as the second wavelength λ2.

The wavelength band WL is defined such that an absorption intensity in the ¹³CO₂ is obtained that is comparatively close to an absorption intensity of the ¹²CO₂. Thus, the amounts of these carbon dioxides can be detected with high accuracy.

In the embodiment, the range of the wavelength band WL is, for example, preferably not less than 4.3478 μm and not more than 4.3804 μm (that is, not less than 2281 cm⁻¹ and not more than 2300 cm⁻¹). The range of the wavelength band WL is, for example, more preferably not less than 4.3535 μm and not more than 4.3573 μm (that is, not less than 2295 cm⁻¹ and not more than 2297 cm⁻¹).

A center value of the wavelength of the measurement light 30L is, for example, not less than 4.3535 micrometers (μm) and not more than 4.3573 μm. A difference between a maximum value of the wavenumber of the wavelength band WL and a minimum value of the wavenumber of the wavelength band WL is, for example, not less than 0.2 cm⁻¹ and not more than 5 cm⁻¹. The difference is, for example, about 1 cm⁻¹.

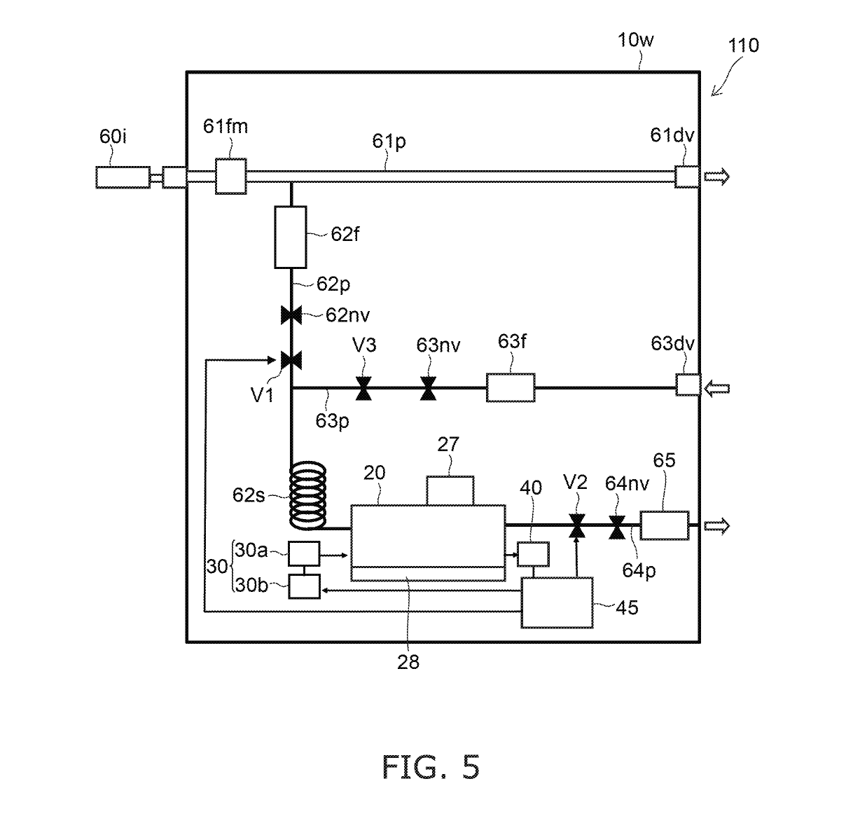

FIG. 5 is a schematic view illustrating the exhalation diagnostic device according to the first embodiment.

As illustrated in FIG. 5, the exhalation diagnostic device 110 is provided with an enclosure 10w. The cell portion 20, the light source 30, the detector 40, and the controller 45 are provided within the enclosure 10w. The controller 45 may be provided outside the enclosure 10w.

A gas introduction part 60i is connected to the enclosure 10w. The gas introduction part 60i is, for example, a mouthpiece. A cannula tube or the like may be used for the gas introduction part 60i. A mask may be used for the gas introduction part 60i.

A first pipe 61p is provided within the enclosure 10w. A first end of the first pipe 61p is connected to the gas introduction part 60i. A second end of the first pipe 61p is connected to the external environment. In this example, a flowmeter 61fm is provided on an entrance side of the first pipe 61p. The flowmeter 61fm is connected to the gas introduction part 60i. A one-way valve 61dv is provided on an exit side of the first pipe 61p. A portion of the sample gas 50 introduced from the gas introduction part 60i is released to the external environment via the one-way valve 61dv.

A second pipe 62p is connected to the first pipe 61p. A first end of the second pipe 62p is connected to the first pipe 61p and a second end of the second pipe 62p is connected to the cell portion 20. In this example, a dehumidifying unit 62f is provided on the path of the second pipe 62p. A filter for absorbing water or the like, for example, is used as the dehumidifying unit 62f. The first valve V1 (solenoid valve) is provided between the first pipe 61p and the cell portion 20. In this example, a needle valve 62nv is provided between the first valve V1 and the dehumidifying unit 62f. In this example, spiral tubing 62s is provided between the first valve V1 and the cell portion 20. The spiral tubing 62s may be omitted. The needle valve 62nv may be provided or omitted as necessary.

The cell portion 20 may, for example, be provided with a heater 28. The cell portion 20 may, for example, be provided with a pressure gauge 27.

A first end of a third pipe 63p is connected at a portion between the first valve V1 and the spiral tubing 62s. A second end of the third pipe 63p is connected to a one-way valve 63dv. The third pipe 63p can introduce air from the external environment to the cell portion 20. A third valve V3 (solenoid valve) is provided in the third pipe 63p. A CO₂ filter 63f is provided between the third valve V3 and the one-way valve 63dv. The CO₂ filter 63f reduces the amount of carbon dioxide in the air introduced from the external environment. In this example, a needle valve 63nv is provided between the third valve V3 and the CO₂ filter 63f. Air is introduced from the external environment via the one-way valve 63dv. CO₂ is removed from the air layer by the air being passed through the CO₂ filter. The air from which CO₂ has been removed passes through the third valve V3 and can be introduced into the cell portion 20. The needle valve 63nv may be provided or omitted as necessary.

Due to the operations of the valve, the sample gas 50 passes through the second pipe 62p and is introduced into the cell portion 20. Alternatively, air from which CO₂ has been removed passes through the third pipe 63p and is introduced into the cell portion 20.

A first end of a fourth pipe 64p is connected to an exit side of the cell portion 20. A second end of the fourth pipe 64p leads to the external environment (outside of the enclosure 10w). In this example, a second valve V2 (solenoid valve) is provided in the fourth pipe 64p. A discharge unit 65 (pump, fan, or the like) is provided between the second valve V2 and the external environment. In this example, a needle valve 64nv is provided between the discharge unit 65 and the second valve V2. The needle valve 64nv may be provided or omitted as necessary.

That is, a portion of the sample gas 50 introduced from the gas introduction part 60i is introduced to the cell portion 20 via the second pipe 62p. The first substance 51 and the second substance 52 in this gas (the exhaled air 50a) are detected in the cell portion 20.

Another portion (most) of the sample gas 50 introduced from the gas introduction part 60i is released to the external environment via the first pipe 61p. That is, the amount (flow rate) of the sample gas 50 flowing through the first pipe 61p is greater than the amount (flow rate) of the sample gas 50 flowing through the second pipe 62p. As such, hardship felt by a subject (human) when collecting the sample gas 50 is suppressed.

By using the flowmeter 61fm, the state of introduction of the sample gas 50 can be detected. Detection operations are performed on the basis of the results of this detection. That is, the introduction start of the sample gas 50 becomes clear and the accuracy of the detection is improved.

By using the needle valve 62nv, the flow rate in the second pipe 62p is restricted and, thus, a stable supply of the sample gas 50 is possible.

By setting the first valve V1 to the open state, the sample gas 50 is introduced into the cell portion 20. The first valve V1 and the second valve V2 are set to the closed state during the detection of the first substance 51 and the second substance 52 in the sample gas 50 that has been introduced into the cell portion 20 (that is, in the sample data measurement period Ps1). Thus, the state of the gas in the cell portion 20 stabilizes and the operations of detection are enhanced. In the sample data measurement period Ps1, the third valve V3 is set to the closed state.

It is preferable that a temperature of the sample gas 50 introduced to the cell portion 20 is constant. By using the spiral tubing 62s, the heater, and the like, the temperature of the sample gas 50 introduced into the cell portion 20 can be controlled with high accuracy. The temperature is, for example, about 40° C.

By setting the third valve V3 to the open state and operating the second valve V2, the needle valve 64nv, and the discharge unit 65, the gas in the cell portion 20 can be released to the external environment.

When performing the detection operations in a state where the sample gas 50 has not been introduced into the cell portion 20 (that is, the reference data measurement period Pr1), the first valve V1 is set to the closed state, and the second valve V2 and the third valve V3 are set to the open state. Thus, air (air from which CO₂ has been removed) is introduced into the cell portion 20 from the external environment.

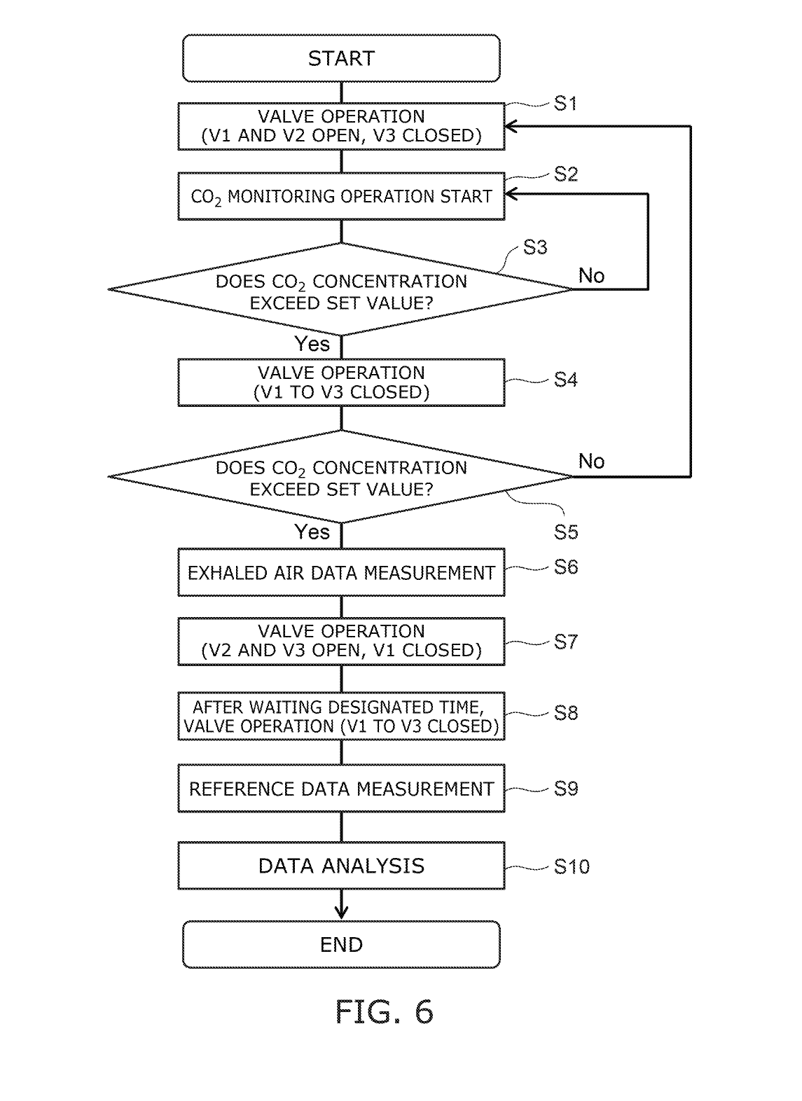

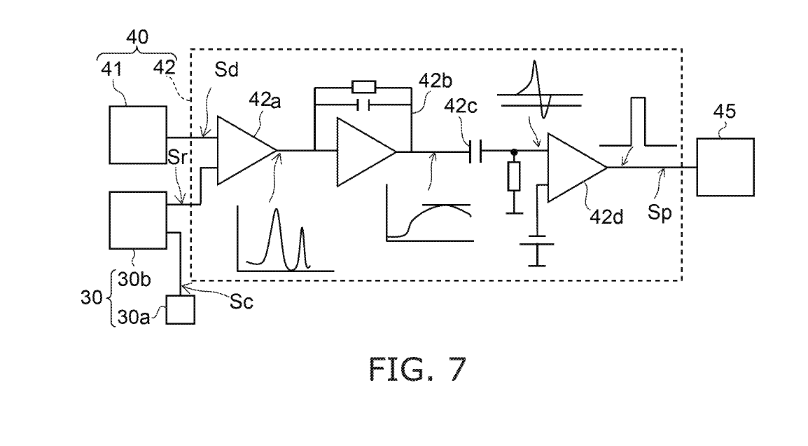

FIG. 6 is a schematic view illustrating the operations of the exhalation diagnostic device according to the first embodiment.

FIG. 6 illustrates an example of operations of a case where the exhalation diagnostic device 110 performs an operation as a capnometer (the second operation OP2) and a CO₂ isotope ratio measurement operation (the first operation OP1).

Measurement is started. First, the valves are operated (step S1). Specifically, the first valve V1 and the second valve V2 are set to the open state, and the third valve V3 is set to the closed state.

Then, monitoring of CO₂ concentration is performed (step S2). This operation corresponds to the second operation OP2.

Determination is performed as to whether the concentration of the CO₂ exceeds a set value (e.g. a predetermined value) (step S3). When the concentration of the CO₂ does not exceed the set value in step S3, return to step S2. When the concentration of the CO₂ exceeds the set value in step S3, proceed to step S4 below. Note that in the determination of step S3, any of the first standard, the second standard, or the third standard described above may be used.

When the concentration of the CO₂ exceeds the set value, the valves are operated (step S4). Specifically, the first valve V1, the second valve V2, and the third valve V3 are set to the closed state.

Determination is performed as to whether the concentration of the CO₂ exceeds a set value (e.g. a predetermined value) (step S5). When the concentration of the CO₂ does not exceed the set value in step S5, return to step S1. When the concentration of the CO₂ exceeds the set value in step S5, proceed to step S6 below. Note that in the determination of step S5, any of the first standard, the second standard, or the standard described above may be used.

Exhaled air data is measured (step S6).

The valves are operated (step S7). Specifically, the second valve V2 and the third valve V3 are set to the open state, and the first valve V1 is set to the closed state. After waiting a specified amount of time, the valves are operated (step S8). Specifically, the first valve V1, the second valve V2, and the third valve V3 are set to the closed state.

Thereafter, reference data is measured (step S9). Then, data analysis is performed (step S10). This operation corresponds to the first operation OP1, and completes the measurement. Note that the order of the exhaled air data measurement of steps S1 to S6 and the reference data measurement of steps S7 to S9 may be interchanged.

### Second Embodiment

FIG. 7 is a schematic view illustrating an exhalation diagnostic device according to a second embodiment.

FIG. 7 illustrates the detector 40.

As illustrated in FIG. 7, the detector 40 is provided with the detection device 41 and the circuit portion 42. As described previously, light, that has passed through the space 23s in which the sample gas 50 is introduced, enters the detection device 41. The detection device 41 outputs a detection signal Sd corresponding to the intensity of that light. The detection signal Sd is input to the circuit portion 42, and a predetermined processing is performed in the circuit portion 42. A processed signal Sp that has been subjected to the processing is supplied to the controller 45. In this example, for example, in the second operation OP2, the wavelength (the third wavelength λ3) of the measurement light 30L is swept.

In this example, the circuit portion 42 is provided with a differential amplifier circuit 42a, an integrator circuit 42b, a differentiator circuit 42c, and a comparing circuit 42d. The detection signal Sd of the detection device 41 is input to a first input of the differential amplifier circuit 42a. A reference signal Sr output from the driving portion 30b of the light source 30 is input to a second input of the differential amplifier circuit 42a.

On the other hand, in the light source 30, a control signal Sc is output from the driving portion 30b to the light emitting portion 30a. The wavelength of the light is changed by this control signal Sc. That is, in the light source 30, a control signal Sc is provided for controlling the changing of the wavelength of the light. The reference signal Sr described above is linked to this control signal Sc.

Output of the differential amplifier circuit 42a is input to the integrator circuit 42b and subjected to integral processing. Output of the integrator circuit 42b is input to the differentiator circuit 42c and subjected to differential processing. Output of the differentiator circuit 42c is input to the comparing circuit 42d, and a difference with a reference voltage (reference signal) is output as the processed signal Sp. The processed signal Sp is input to the controller 45.

The circuit portion 42 outputs the processed signal Sp corresponding to the difference between the reference signal Sr and the detection signal Sd output from the detection device 41.

The controller 45, when performing the second operation OP2, performs the detection of the temporal change described above on the basis of the processed signal Sp output from the circuit portion 42.

Thus, in the second operation OP2, a circuit portion 42 that performs analog signal processing can be used. In the light emitting portion 30a, characteristics may vary due to the effects of temperature and the like. Consequently, the wavelength may shift from the target wavelength. In this case, by using the analog circuit according to the embodiment, changes in the characteristics can be compensated for and high-speed processing can be performed. The performance of complex digital data processing can be eliminated and the second operation OP2 can be performed with high accuracy and high speed.

In the embodiment, a change over time of the relative ratio of the ¹²CO₂ to the ¹³CO₂ contained in the exhaled air 50a may be measured. For example, there is a relationship between gastric emptying and the relative amount of ¹³CO₂. Gastric emptying can be diagnosed on the basis of the results of measuring the change over time of the relative ratio of the ¹²CO₂ to the ¹³CO₂.

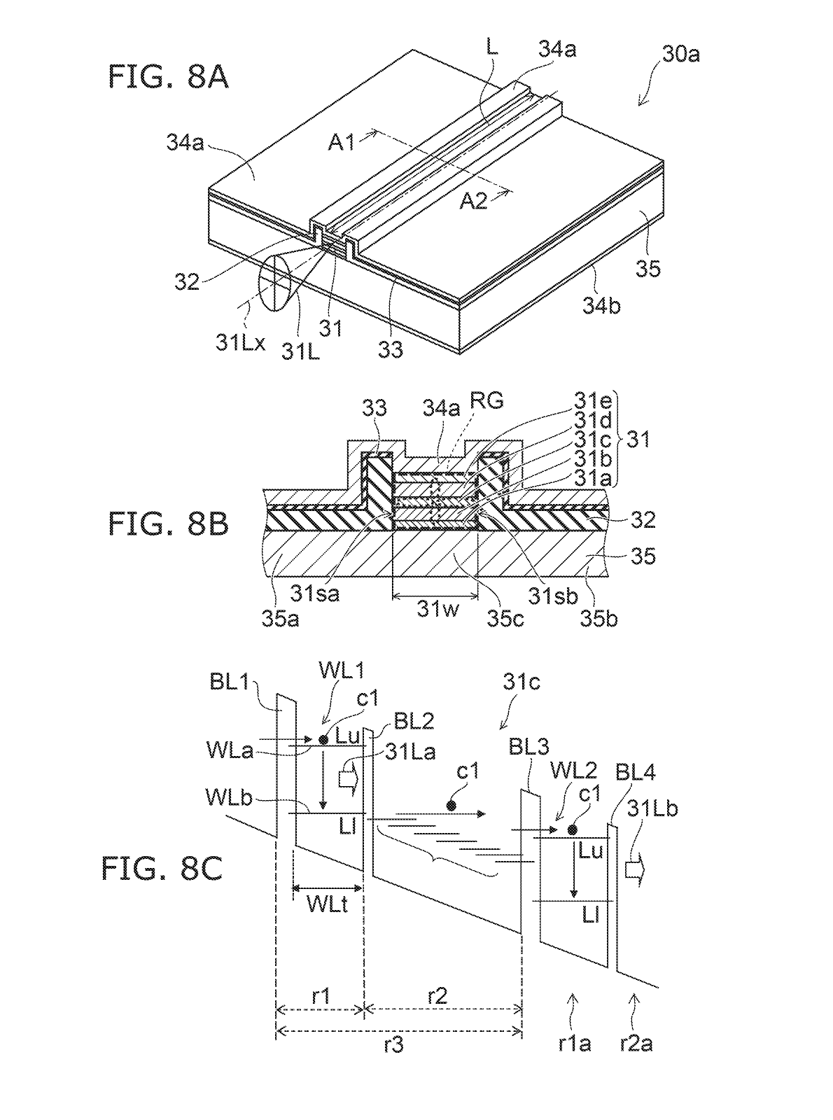

FIGS. 8A to 8C are schematic views illustrating a portion of the exhalation diagnostic device according to an embodiment.

FIG. 8A is a schematic perspective view. FIG. 8B is a cross-sectional view taken along line A1-A2 in FIG. 8A. FIG. 8C is a schematic view illustrating the operations of the light source 30.

In the example, a semiconductor light emitting device 30aL is used as the light source 30. A laser is used as the semiconductor light emitting device 30aL. In this example, a quantum cascade laser is used.

As illustrated in FIG. 8A, the semiconductor light emitting device 30aL includes a substrate 35, a stacked body 31, a first electrode 34a, a second electrode 34b, a dielectric layer 32 (first dielectric layer), and an insulating layer 33 (second dielectric layer).

The substrate 35 is provided between the first electrode 34a and the second electrode 34b. The substrate 35 includes a first portion 35a, a second portion 35b, and a third portion 35c. These portions are disposed in one plane. This plane may intersect or be parallel to a direction from the first electrode 34a toward the second electrode 34b. The third portion 35c is disposed between the first portion 35a and the second portion 35b.

The stacked body 31 is provided between the third portion 35c and the first electrode 34a. The dielectric layer 32 is provided between the first portion 35a and the first electrode 34a and between the second portion 35b and the first electrode 34a. The insulating layer 33 is provided between the dielectric layer 32 and the first electrode 34a.

The stacked body 31 has a stripe shape. The stacked body 31 functions as a ridge waveguide RG. Two edge faces of the ridge waveguide RG are mirror faces. Light 31L emitted in the stacked body 31 is emitted from an edge face (emission face). The light 31L is infrared laser light. An optical axis 31Lx of the light 31L follows an extending direction of the ridge waveguide RG.

As illustrated in FIG. 8B, the stacked body 31 includes, for example, a first cladding layer 31a, a first guide layer 31b, an active layer 31c, a second guide layer 31d, and a second cladding layer 31e. These layers are juxtaposed in this order along the direction from the substrate 35 toward the first electrode 34a. Each of a refraction index of the first cladding layer 31a and a refraction index of the second cladding layer 31e is lower than each of a refraction index of the first guide layer 31b, a refraction index of the active layer 31c, and a refraction index of the second guide layer 31d. The light 31L produced at the active layer 31c is confined in the stacked body 31. In some cases, the first guide layer 31b and the first cladding layer 31a are referred to collectively as a cladding layer. In some cases, the second guide layer 31d and the second cladding layer 31e are referred to collectively as a cladding layer.

The stacked body 31 has a first side face 31sa and a second side face 31sb perpendicular to the optical axis 31Lx. A distance 31w (width) between the first side face 31sa and the second side face 31sb is, for example, not less than 5 μm and not more than 20 μm. As such, for example, control of a horizontal transverse direction mode is facilitated, and improvements in output are facilitated. If the distance 31w is excessively long, higher-order modes will be easily generated in the horizontal transverse direction mode and it will be difficult to increase the output.

A refractive index of the dielectric layer 32 is lower than the refractive index of the active layer 31c. As such, the ridge wave guide RG is formed along the optical axis 31Lx by the dielectric layer 32.

As illustrated in FIG. 8C, the active layer 31c has, for example, a cascade structure and, in the cascade structure, for example, a first region r1 and a second region r2 are alternately stacked. A unit structure r3 includes the first region r1 and the second region r2. A plurality of the unit structure r3 is provided.

For example, a first barrier layer BL1 and a first quantum well layer WL1 are provided in the first region r1. A second barrier layer BL2 is provided in the second region r2. For example, a third barrier layer BL3 and a second quantum well layer WL2 are provided in another first region r1a. A fourth barrier layer BL4 is provided in another second region r2a.

Intersubband optical transitions in the first quantum well layer WL1 occur in the first region r1. Thus, for example, the light 31L of a wavelength of not less than 3 μm and not more than 18 μm is emitted.

Energy of a carrier c1 (e.g. electrons) injected from the first region r1 is relaxable in the second region r2.

A well width WLt in a quantum well layer (e.g. the first quantum well layer WL1) is, for example, not more than 5 nm. When the well width WLt is narrow like this, energy levels are discretized and, for example, a first sub-band WLa (upper energy level Lu), a second sub-band WLb (lower energy level Ll), and the like are generated. The carrier c1 injected from the first barrier layer BL1 is effectively confined in the first quantum well layer WL1.

When the carrier c1 makes a transition from the upper energy level Lu to the lower energy level Ll, a light 31La corresponding to an energy difference (a difference between the upper energy level Lu and the lower energy level Ll) is emitted. That is, optical transitions occur.

In the same manner, in the second quantum well layer WL2 of the other first region r1a, a light 31Lb is emitted.

In the embodiment, the quantum well may include a plurality of wells for which wave functions overlap. The upper energy level Lu of each of the plurality of quantum wells may be the same as each other. The lower energy level Ll of each of the plurality of quantum wells may be the same as each other.

For example, the intersubband optical transitions occur in either a conduction band or a valence band. For example, recombination via a p-n junction of a hole and an electron is not needed. For example, optical transitions occur due to either the hole or the electron carrier c1, and light is emitted.

In the active layer 31c, for example, due to the voltage applied between the first electrode 34a and the second electrode 34b, the carrier c1 (e.g. electrons) is injected into a quantum well layer (e.g. the first quantum well layer WL1) via a barrier layer (e.g. the first barrier layer BL1). Thus, intersubband optical transitions occur.

The second region r2 has, for example, a plurality of sub-bands. The sub-bands are, for example, mini-bands. The energy difference among the sub-bands is small. It is preferable that the sub-bands are nearly a continuous energy band. As a result, the energy of the carrier c1 (electrons) is relaxed.

In the second region r2, for example, light (e.g. infrared light of a wavelength of not less than 3 μm and not more than 18 μm) substantially is not emitted. The carrier c1 (electrons) of the lower energy level Ll of the first region r1 passes through the second barrier layer BL2, is injected into the second region r2, and is relaxed. The carrier c1 is injected into the other cascaded first region r1a. Optical transitions occur in this first region r1a.

In the cascade structure, optical transitions occur in each of the plurality of unit structures r3. Thus, it is easy to obtain high light output in the entirety of the active layer 31c.

As described, the light source 30 includes the semiconductor light emitting device 30aL. The semiconductor light emitting device 30aL emits the measurement light 30L due to the energy relaxation of the electrons in the sub-bands of the plurality of quantum wells (e.g. the first quantum well layer WL1, the second quantum well layer WL2, and the like).

InGaAs, for example, is used for the quantum wells (e.g. the first quantum well layer WL1, the second quantum well layer WL2, and the like). InAlAs, for example, is used for the barrier layers (e.g. the first to fourth barrier layers BL1 to BL4, and the like). Here, for example, if InP is used as the substrate 35, excellent lattice matching in the quantum well layers and the barrier layers can be obtained.

The first cladding layer 31a and the second cladding layer 31e include Si, for example, as an n-type impurity. An impurity concentration in these layers is, for example, not less than 1×10¹⁸ cm⁻³ and not more than 1×10²⁰ cm⁻³ (e.g. about 6×10¹⁸ cm⁻³). A thickness of each of these layers is, for example, not less than 0.5 μm and not more than 2 μm (e.g. about 1 μm).

The first guide layer 31b and the second guide layer 31d include Si, for example, as an n-type impurity. The impurity concentration in these layers is, for example, not less than 1×10¹⁶ cm⁻³ and not more than 1×10¹⁷ cm⁻³ (e.g. about 4×10¹⁶ cm⁻³). A thickness of each of these layers is, for example, not less than 2 μm and not more than 5 μm (e.g. 3.5 μm).

The distance 31w (the width of the stacked body 31, that is, the width of the active layer 31c) is, for example, not less than 5 μm and not more than 20 μm (e.g. about 14 μm).

A length of the ridge wave guide RG is, for example, not less than 1 mm and not more than 5 mm (e.g. about 3 mm). The semiconductor light emitting device 30aL operates at, for example, an operating voltage of not more than 10 V. Consumption current is lower compared to carbon dioxide gas laser devices. As such, low power consumption operation is possible.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the invention

What is claimed is:

1. An exhalation diagnostic device comprising:
a cell portion including space into which a sample gas containing a first substance and a second substance different than the first substance is introduced;
a light source that emits light toward the space;
a detector that detects an intensity of the light transmitted through the space; and
a controller,
the controller, at a time of a first operation, causing the light source to change a wavelength of the light within a wavelength band including a first wavelength of a first peak of light absorption of the first substance, and a second wavelength, differing from the first wavelength, of a second peak of light absorption of the second substance, and calculating a ratio of an amount of the second substance contained in the sample gas to an amount of the first substance contained in the sample gas on a basis of detection results of an intensity of the light of the first wavelength and an intensity of the light of the second wavelength detected by the detector, and
the controller, at a time of a second operation performed in one respiration, causing the light source to set the wavelength of the light to a third wavelength, determining whether concentration of at least one of the first substance and the second substance exceeds a set value or not on a basis of detection results of the intensity of the light of the third wavelength detected by the detector, and starting the first operation when the concentration exceeds the set value.

2. The device according to claim 1, wherein:
the first substance is carbon dioxide including ¹²C; and
the second substance is carbon dioxide including ¹³C.

3. The device according to claim 2, wherein the third wavelength is the same as the first wavelength.

4. The device according to claim 2, wherein the third wavelength is the same as the second wavelength.

5. The device according to claim 2, wherein each of the first wavelength and the second wavelength is not less than 4.345 micrometers and not more than 4.384 micrometers.

6. The device according to claim 1, wherein the controller performs the first operation on a basis of detection results of a temporal change in the amount of one of the first substance and the second substance.

7. The device according to claim 1, wherein:
the detector further includes:
a detection device into which the light transmitted through the space is introduced, and which outputs a detection signal corresponding to the intensity of the light; and
a circuit portion that outputs a processed signal corresponding to a difference between the detection signal output from the detection device and a reference signal, and
the controller, at a time of the second operation, performs the detection of the temporal change on a basis of the processed signal output from the circuit portion.

8. The device according to claim 7, wherein the detection device includes a semiconductor sensor device.

9. The device according to claim 7 wherein the reference signal is linked to a control signal that controls the changing of the wavelength of the light of the light source.

10. The device according to claim 1, wherein
the light source includes:
a semiconductor light emitting device that emits luminescent light as a result of energy relaxation of electrons in a plurality of sub-bands of quantum wells; and
a wavelength controller that adjusts a wavelength of the luminescent light so as to generate light.

11. The device according to claim 1, wherein a difference between a maximum value of a wavenumber of the wavelength band and a minimum value of the wavenumber of the wavelength band WL is not less than 0.2 cm⁻¹ and not more than 5 cm⁻¹.
